(19) United States
(12) Patent Application Publication
(10) Pub. No.: US 2026/0273108 A1
(43) Pub. Date: Sep. 17, 2026

(54) USE OF ACRYLIC ACID-CONTAINING POLYMERS FOR MOLECULAR IMAGING OF TARGET PROTEINS

(71) Applicant: The Johns Hopkins University, Baltimore, MD (US)

(72) Inventors: Aline Thomas, Baltimore, MD (US); Peter van Zijl, Baltimore, MD (US)

(21) Appl. No.: 19/670,307

(22) Filed: May 7, 2026

Related U.S. Application Data
(63) Continuation of application No. PCT/US2024/053322, filed on Oct. 29, 2024.
(60) Provisional application No. 63/597,712, filed on Nov. 10, 2023.

(51) Int. Cl. A61K 49/12; A61K 49/08

(52) U.S. Cl. CPC A61K 49/126; A61K 49/085; A61K 2123/00

(57) ABSTRACT

Acrylic acid-containing polymers that can be used as MRI agents, and condensation reactions for producing same is described herein. Condensation reactions can be used to attach a number of MRI signal-generating molecules and targeting molecules to the polymer, wherein the targeting molecule binds a molecular target of interest such as a cancer biomarker. Advantageously, the acrylic acid-containing polymers provide multiple attachment sites for the MRI signal-generating molecule, enabling an increase in the number of less toxic and less immunogenic MRI signal-generating molecules to detectable levels.

### CROSS-REFERENCE TO RELATED APPLICATIONS

This application claims the benefit of U.S. Provisional Application No. 63/597,712, filed Nov. 10, 2023, the contents of which are incorporated herein by reference in its entirety.

### GOVERNMENT SUPPORT CLAUSE

This invention was made with government support under grant EB030612 awarded by the National Institutes of Health. The government has certain rights in the invention.

### FIELD

The present disclosure relates to molecular imaging agents that can be visualized using magnetic resonance imaging (MRI).

### BACKGROUND

The low chemical sensitivity of MRI has been a persistent obstacle for the development of molecular imaging agents that can be visualized using MRI scanners. These agents were often designed “one-to-one,” wherein one targeting molecule is attached to one signal-generating molecule, often with a linker. This monomeric design requires molecules that can generate strong MR signals in order to detect protein targets, which are often present in the micro- to milli-Molar range. Disadvantageously, signal generating molecules in the current art with these attributes (e.g., gadolinium and manganese) are too toxic or immunogenic at detectable doses. To overcome this challenge, polymers have recently been incorporated into the design of molecular MRI agents. The repeated chemical groups of polymers can provide multiple attachment sites for the signal-generating molecule, enabling an increase in the number of less toxic and less immunogenic MRI signal-generating molecules to detectable levels. Previously, the proof-of-concept of polymeric molecular MRI agents has previously been demonstrated with two formulations that could detect prostate-specific membrane antigen (PSMA) using the chemical exchange saturation transfer (CEST) MRI method [Banerjee, 2018; Liu, 2017]. Still, the translation and widespread clinical use of polymeric molecular MRI agents require improvements to their sensitivity and specificity. For example, physiochemical properties (i.e., charge, hydrophobicity) have been shown to influence the in vitro and in vivo specificity of molecular imaging agents.

There continues to be a need in the art for MRI agents, e.g., CEST MRI agents, that can be designed to be specific and sensitive to a target of interest while simultaneously being substantially nontoxic. The designer MRI agents described herein can be internalized, and thus better retained, in a tumor cell, and can be imaged with minimal toxicity to the subject because they comprise naturally occurring components and/or have high safety profiles.

### SUMMARY

In some aspect, the present invention relates to a polymeric molecular MRI agent comprising a polymer and at least one signal-generating molecule.

In some other aspects, the present invention relates to a polymeric molecular MRI agent comprising a polymer, at least one signal-generating molecule, and at least one targeting molecule.

In still other aspects, the present invention relates to a polymeric molecular MRI agent comprising a polymer, at least one signal-generating molecule, at least one targeting molecule, and at least one therapeutic agent.

In some embodiments, the at least one signal-generating molecule comprises alanine, 4-aminosalicyclic acid, arginine, barbituric acid, glucosamine, glutamine, uridine, or any combination thereof.

In some embodiments, the polymer is an acrylic-acid containing polymer.

In some embodiments, the at least one targeting molecule binds a molecular target of interest. In some embodiments, the molecular target of interest is a cancer biomarker.

In other aspects, the present invention relates to a method of making the polymeric molecular MRI agents described herein, said method comprising:
dissolving a polymer in a first water-miscible solution to produce a first solution;
adding an amount of 1-ethyl-3-(3-dimethylaminopropyl)carbodiimide (EDC) and an amount of N-hydroxysuccinimide (NHS) to the first solution to produce a second solution;
dissolving at least one signal-generating molecule and at least one targeting molecule to a second water-miscible solution to produce a third solution; and
combining the second solution and the third solution,
   wherein the at least one signal-generating molecule and the at least one targeting molecule are conjugated to the polymer using a carbodiimide reaction to produce the polymer molecular MRI agent.

In yet another aspect, the present invention relates to a method of determining if a subject has a tumor, said method comprising administering a bolus of a polymer molecular MRI agent described herein to a subject suspected of having a tumor, and imaging a region of the subject to determine if the polymer molecular MRI agent has been internalized and biodistributed in cells of the tumor, wherein the polymer molecular MRI agent comprises a targeting molecule specific to a molecular target in the cells of the tumor.

In other aspects, the present invention relates to a method of monitoring a change in size of a known tumor in a subject, said method comprising administering a bolus of a polymer molecular MRI agent described herein to the subject with the known tumor, and imaging a region of the subject in proximity of the known tumor to determine if the tumor has increased or decreased in size, wherein the polymer molecular MRI agent comprises a targeting molecule specific to a molecular target in the known tumor.

In another aspect, the present invention relates to a method of using a polymer molecular MRI agents described herein for measurement of pH and evaluation of pH changes, in vivo, wherein said method comprises introducing (i) multiple different polymer molecular MRI agents whose pH sensitivity for signal generation differ or (ii) polymer molecular MRI agents with multiple different signal-generating molecules with differing pH sensitivity to a subject, and imaging a region in proximity of where the polymer molecular MRI agents have been introduced with CEST MRI, wherein the CEST MRI images permit measurement of pH and the evaluation of its changes.

In yet another aspect, the present invention relates to a method of using a polymer molecular MRI agents described herein for theranostic imaging in a subject, said method comprising administering a bolus of the polymer molecular MRI agent to the subject with a known tumor, and imaging a region of the subject in proximity of the known tumor, wherein the polymer molecular MRI agent comprises a targeting molecule specific to a molecular target in the known tumor.

Other aspects, features and advantages of the invention will be more fully apparent from the ensuing disclosure and appended claims.

### BRIEF DESCRIPTION OF THE FIGURES

FIG. 1. UV-VIS spectroscopy of polymer molecular MRI agents described herein in phosphate buffered saline (PBS), i.e., p (signal-generating molecule), compared to that of PAA mixed with the signal-generating molecule, i.e., PAA+signal-generating molecule, and to that of the signal-generating molecule in absence of PAA.

FIG. 2. NMR of polymer molecular MRI agents in PBS, i.e., p(signal-generating molecule), compared to that of only PAA. Solutions were 10 mM for Uridine, 5 mM for Barbituric Acid, else 20 mM.

FIG. 3. CEST MRI of polymer molecular MRI agents in PBS, i.e., p(signal-generating molecule), compared to that of PAA mixed with the signal-generating molecules, i.e., PAA+signal-generating molecule, to that of the signal-generating molecule in absence of PAA and to that of PBS alone. Solutions were 10 mM for Uridine, 5 mM for Barbituric Acid, else 20 mM. The so-called Z-spectra is the reduction of the normalized water signal intensity (S/S₀) displayed as a function of RF saturation frequency offset (Δω) relative to the water protons, where S is the MRI signal in the presence of a saturation RF pulse and S₀ is the MRI signal in the absence of a saturation RF pulse.

### DETAILED DESCRIPTION OF THE DISCLOSURE

Although the claimed subject matter will be described in terms of certain embodiments, other embodiments, including embodiments that do not provide all of the benefits and features set forth herein, are within the scope of this disclosure as well. Various structural and parameter changes may be made without departing from the scope of this disclosure.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

“About” and “approximately” are used to provide flexibility to a numerical range endpoint by providing that a given value may be “slightly above” or “slightly below” the endpoint without affecting the desired result, for example, +/−5%.

The phrase “in one embodiment” or “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, a “system” refers to a plurality of real and/or abstract elements operating together for a common purpose. In some embodiments, a “system” is an integrated assemblage of hardware and/or software elements. In some embodiments, each component of the system interacts with one or more other elements and/or is related to one or more other elements. In some embodiments, a system refers to a combination of components and software for controlling and directing methods.

As used herein, the term “substantially free,” when used in the context of a composition or component of a composition that is substantially absent, is intended to refer to an amount that is less then about 2% by weight, e.g., less than about 1% by weight, less than about 0.5% by weight, less than about 0.1% by weight, less than about 0.05% by weight, or less than about 0.01% by weight of the stated material, based on the total weight of the composition.

As introduced hereinabove, incorporating polymers into imaging agents can facilitate the use of less toxic MRI contrast materials and tracers with lower detection sensitivity. The repeated functional groups on polymers provide multiple attachment sites, which enables an increase in the number of contrast materials and tracers in the MRI agents. Without being bound by theory, it is believed that a polymer can be used to summate the signal of less toxic molecules to a more optimal level of detection. For example, prostate-specific membrane antigen (PSMA)-targeting agents, which were first developed and translated for clinical use in positron emission tomography (PET) imaging [Szabo, 2015], were successfully adapted for MRI using polymers to link PSMA ligands to MRI-sensitive compounds [Liu, 2017; Banerjee, 2018]. Disadvantageously, the widespread use of polymeric molecular MRI agents will require improvements in sensitivity and specificity. Differences in the in vivo signal dynamics of these PSMA-targeting CEST MRI agent formulations [Id.] may be due to their differences in hydrophilicity and charge. Further, the hydrophilicity [Zanetti-Domingues, 2013; Wüstermann, 2016; Benešová, 2016] and charge [Numasawa, 2020; García Garayoa, 2008] of the imaging agent can influence in vitro and in vivo specificity, as well as circulation time [García Garayoa, 2008] and tumor cell retention [Benešová, 2016; García Garayoa, 2008]. Complicating the widespread use of polymeric molecular MRI agents is the fact that these physiochemical properties (e.g., size, spacing, charge, hydrophilicity) are not independent of each other.

Chemical exchange saturation transfer (CEST) is an MRI method that can facilitate the use of more biocompatible organic compounds [van Zijl, 2011] to serve as the signal-generating molecule in molecular MRI agents. A radiofrequency pulse can be applied to saturate their protons. These protons can then exchange with those on water, transferring their saturation to the water molecules that affect the MRI signal on proton (¹H) scanners. Repeating these processes can amplify the effect, permitting indirect detection of compounds present at micro- to milli-Molar concentrations with a sensitivity in the Molar range. Through this mechanism, many molecules can be monitored using ¹H MRI scanners indirectly through their water-exchanging protons. Advantageously, maps of CEST contrast can be obtained in patients with cancer using standard MRI equipment available in hospitals. Further, CEST MRI is being developed and clinically tested by groups world-wide for a range of applications.

In a first aspect, the present invention relates to polymeric molecular MRI agents comprising a polymer, at least one signal-generating molecule, and optionally, at least one targeting molecule. In some embodiments, the polymeric molecular MRI agents comprise a polymer and at least one signal-generating molecule, and hemodynamic imaging can be performed. In some embodiments, the polymeric molecular MRI agents comprise a polymer, at least one signal-generating molecule, and at least one targeting molecule. In some embodiments, the polymer is a multivalent linker, which permits the linkage of multiple signal-generating molecules to a targeting molecule, which advantageously increases the signal generated by the molecular MRI agent to detectable levels for the target of interest. The biocompatible signal-generating molecules can be detected through their water-exchanging protons using chemical exchange saturation transfer (CEST), a molecularly sensitive MRI method. NMR and UV-VIS spectra demonstrated carbodiimide chemistry can conjugate at least 70% of the polymer's attachment sites with these signal-generating molecules.

Since multiple copies of the signal-generating molecule can be needed for detection of the imaging agent using MRI [Liu, 2017; Banerjee, 2018; Han, 2019], modifying the signal-generating molecule is more practical than modifying the targeting molecule or polymer. In some embodiments, the at least one signal-generating molecule comprises alanine, 4-aminosalicyclic acid, arginine, barbituric acid, glucosamine, glutamine, uridine, or any combination thereof.

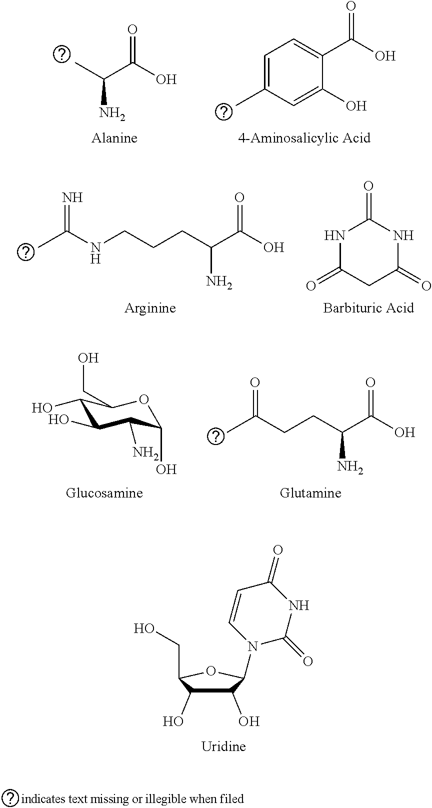

The reported properties of these signal-generating molecules are included in Table 1.

TABLE 1
Biocompatibility upon bolus intravenous (IV) injection in humans.

| | | | Radio | Exchange | | |
|---|---|---|---|---|---|---|
| Signal-generating molecule | | IV Dose | frequency | rate | Properties | |
| Name | Abbr. | mmole/kg | ppm | kₑₓ (s⁻¹) | M.W. | Net |
| Alanine | ALA | 5.61 | 3.0 | 7,670 | 89.1 | 0 |
| 4-Aminosalicylate | ASA | 2.33 | 3.2, 9.5 | N/A, 1,200 | 153.1 | 0 |
| Arginine | ARG | 2.49 | 1.8-2.0 | 1,200 | 174.2 | +1 |
| Barbituric acid | BA | 1.56* | 5.0 | N/A | 127.1 | −1 |
| Glucosamine | GA | 1.86 | 1.2 | 2,900-14,000^{#} | 179.2 | +1 |
| Glutamine | GLN | 5.88 | 2.1, 2.8-2.9 | 12,600 | 146.2 | 0 |
| Uridine | UR | 1.43 | 6.3-6.4 | 3080 | 244.2 | −1 |

*= IV dose for rodents.
CEST properties include proton exchange rate and radiofrequency for saturation.
Exchange rate (kₑₓ) for water-exchanging protons at a pH of 7-7.4 and temperature of 36-67° C.
^{#}= exchange rates for glucose.
Physiochemical properties include molecular weight (M.W.) and net charge.

In some embodiments, at least one signal-generating molecule is conjugated to an acrylic-acid containing polymer. In some embodiments, the acrylic-acid containing polymer is poly(acrylic acid) (PAA) polymer. In some embodiments, commercially available PAA polymers are used, wherein the MW is in a range from about 1250 to about 20000 Da. In some embodiments, the MW of the PAA polymers is in a range from about 1250 to about 10000 Da, or about 1250 to about 5000 Da, or about 1250 to about 2500 Da, or about 1500 to about 2500 Da. In some other embodiments, the MW of the PAA polymers is in a range from about 10 to about 12 kDa, or about 12 to about 14 kDa, or about 14 to about 16 kDa, or about 16 to about 18 kDa, or about 18 to about 20 kDa. In some embodiments, the acrylic-acid containing polymer is poly(methacrylic acid). In some other embodiments, the polymer is poly(maleic acid). In some embodiments, at least one signal-generating molecule is conjugated to a poly(acrylic acid) polymer using 1-ethyl-3-(3-dimethylaminopropyl)carbodiimide/N-hydroxysuccinimide (EDC/NHS) chemistry. In some embodiments, the efficiency of conjugation of at least one signal-generating molecule to a PAA polymer is at least about 60%, at least about 65%, or at least about 70. In some embodiments, the mole ratio of the at least one signal-generating molecule to polymer is in a range from about 1:5 to about 1:60 when using PAA polymers having a MW in a range of about 1250 Da to about 20000 Da, wherein the mole ratio is chosen to optimize the physiochemical properties of the signal-generating molecule on said polymeric molecular MRI agents. It should be appreciated that in some embodiments, the polymeric molecular MRI agents comprise only one type of signal-generating molecule (e.g., one of alanine, 4-aminosalicyclic acid, arginine, barbituric acid, glucosamine, glutamine, or uridine) conjugated to the polymer, while in some other embodiments, the polymeric molecular MRI agent comprises a mixture of at least two or more of the signal-generating molecules conjugated to the polymer, as readily understood by the person skilled in the art.

In some embodiments, the at least one signal-generating molecule is selected based on whether the polymeric molecular MRI agents will be detected in an intracellular or extracellular cancer environment. For example, extracellular cancer environments tend to be more acidic and therefore a signal-generating molecule is preferably chosen that generates a strong signal in acidic environments. In some embodiments, the polymeric molecular MRI agents are intended to be taken up and internalized by cells so the signal-generating molecule is chosen to generate a strong signal at intracellular pH values.

In some embodiments, the at least one targeting molecule chosen for the polymeric molecular MRI agents is known to bind to a molecular target of interest, for example a cancer biomarker. For example, in some embodiments, the molecular target will be prostate-specific membrane antigen (PSMA) and the targeting molecule comprises glutamate-ureido-lysine (GUL), which is a PSMA-targeting imaging agent. In some embodiments, the mole ratio of the at least one targeting molecule to polymer is in a range from about 1:1 to about 4:1. Imaging PSMA has been well demonstrated in patients with prostate cancer but is also expected to be broadly useful as it is expressed in the endothelia of a multitude of aggressive cancers including lung and breast cancer [Fragomeni, 2018]. Advantageously, the imaging platform comprising the polymeric molecular MRI agents described herein can be readily applied to image key molecules of the nearly 150 targeted therapies currently approved for cancer applications (cancer dot gov) by interchanging the targeting molecule. In some embodiments, the molecular target of interest comprises the programmed death-ligand 1 (PD-L1) biomarker. In some embodiments, the molecular target of interest comprises arginine-glycine-aspartate (RGD)-binding integrins. In some embodiments, the molecular target of interest comprises Fibroblast Activation Protein-Alpha (FAP).

In some embodiments, the polymeric molecular MRI agents further comprise at least one therapeutic agent, wherein the therapeutic agent is a targeting molecule or is a signal-generating molecule or wherein the therapeutic agent is incorporated into the polymer molecular MRI agent along with the at least one targeting molecule or the at least one signal-generating molecule. In some embodiments, the therapeutic agent comprises a prodrug. The inclusion of the therapeutic agent permits theranostic imaging.

In some embodiments, the polymeric molecular MRI agents are substantially devoid of glucose-rich dextran and maleic anhydride-rich polymers and are substantially devoid of metallic or radioactive labels.

In a second aspect, a method of making a polymeric molecular MRI agent is described, said method comprising:
dissolving a polymer in a first water-miscible solution to produce a first solution;
adding an amount of 1-ethyl-3-(3-dimethylaminopropyl)carbodiimide (EDC) and an amount of N-hydroxysuccinimide (NHS) to the first solution to produce a second solution;
dissolving at least one signal-generating molecule and at least one targeting molecule to a second water-miscible solution to produce a third solution; and
combining the second solution and the third solution, wherein the at least one signal-generating molecule and the at least one targeting molecule are conjugated to the polymer using a carbodiimide reaction to produce the polymer molecular MRI agent.

In some embodiments of the method of the second aspect, the water-miscible solution comprises water and at least one additional species that is water miscible such as dimethylsulfoxide (DMSO) and/or dimethylformamide (DMF). In some embodiments, the first water-miscible solution comprises the same species as the second water-miscible solution, in the same ratios. In some embodiments, the first water-miscible solution comprises the same species as the second water-miscible solution, but in different ratios. In some embodiments, the first water-miscible solution comprises different species than the second water-miscible solution. In some embodiments, the second solution and the third solution are combined at room temperature and stirred. In some embodiments, after production of the polymer molecular MRI agent, impurities are separated from the polymer molecular MRI agent, e.g., using dialysis. It should be appreciated that when the polymer molecular MRI agent doesn't include at least one targeting molecule (e.g., for hemodynamic imaging), that the at least one targeting molecule is not included in the third solution. In some embodiments, when at least one therapeutic agent is incorporated into the polymer molecular MRI agent along with the at least one targeting molecule and/or the at least one signal-generating molecule, the at least one therapeutic agent is also dissolved in the third solution.

In a third aspect, a method of determining if a subject has a tumor is described, said method comprising administering a bolus of the polymer molecular MRI agent of the first aspect to a subject suspected of having a tumor, and imaging a region of the subject to determine if the polymer molecular MRI agent has been internalized and biodistributed in cells of the tumor, wherein the polymer molecular MRI agent comprises a targeting molecule specific to a molecular target in the cells of the tumor. In some embodiments, the polymer molecular MRI agents are not substantially retained in non-cancerous organs or cells in vivo. In some embodiments, the tumor is a prostate tumor. In some embodiments, the tumor is a prostate tumor and the target is a PSMA+tumor, wherein the polymer molecular MRI agent is internalized and retained in the cells of a PSMA+tumor more substantially than in a PSMA-tumor and non-cancerous organs in vivo. In some embodiments, the necessity to perform the method of the third aspect is prompted by assay or other test results, for example, elevated levels of a known cancer biomarker, in said subject. In some embodiments, the assay is a prostate-specific antigen (PSA) test. It should be appreciated that the method of the third aspect can be used to determine if the subject has a tumor where previously there was none or to determine if the subject has a recurring tumor. In some embodiments, the bolus of the polymer molecular MRI agent is intravenously injected into the subject. In some other embodiments, the bolus of the polymer molecular MRI agent is intratumorally injected into the subject. In some other embodiments, the bolus of the polymer molecular MRI agent is intraperitoneally injected into the subject. In some embodiments, the subject is imaged using MRI. In some embodiments, the subject is imaged using CEST MRI. In some embodiments, the region that the subject is imaged is in proximity of the suspected tumor site.

In a fourth aspect, a method of monitoring a change in size of a known tumor in a subject is described, said method comprising administering a bolus of the polymer molecular MRI agent of the first aspect to the subject with the known tumor, and imaging a region of the subject in proximity of the known tumor to determine if the tumor has increased or decreased in size, wherein the polymer molecular MRI agent comprises a targeting molecule specific to a molecular target in the known tumor. In some embodiments, the polymer molecular MRI agents are not substantially retained in non-cancerous organs or cells in vivo. The method of the fourth aspect is particularly useful in the monitoring of a cancer treatment, e.g., chemotherapeutic and/or radiotherapeutic treatment, to determine if the treatment is effective and/or if the treatment options (e.g., dosage amounts and/or dosage frequency) should be adjusted or modified. In some embodiments, the bolus of the polymer molecular MRI agent is intravenously, intratumorally, or intraperitoneally injected into the subject. In some embodiments, the subject is imaged using MRI. In some embodiments, the subject is imaged using CEST MRI. In some embodiments, the region that the subject is imaged is in proximity of the known tumor site.

In a fifth aspect, a method of using the polymer molecular MRI agents of the first aspect for MRI-guided drug delivery is described, said method comprising administering a bolus of the polymer molecular MRI agent of the first aspect to the subject, and imaging a region of the subject in proximity of where the drug is to be delivered, wherein the polymer molecular MRI agent comprises a targeting molecule specific to a molecular target in a region wherein the drug is to be delivered, e.g., a tumor site. In some embodiments, the polymer molecular MRI agents are not substantially retained in non-cancerous organs or cells in vivo. In some embodiments, a bolus of the polymer molecular MRI agent is intravenously, intratumorally, or intraperitoneally injected into the subject.

In a sixth aspect, a method of using the polymer molecular MRI agents of the first aspect for measurement of pH and evaluation of pH changes, in vivo, is described, wherein said method comprises introducing (i) multiple different polymer molecular MRI agents whose pH sensitivity for signal generation differ or (ii) polymer molecular MRI agents with multiple different signal-generating molecules with differing pH sensitivity to a subject, and imaging a region in proximity of where the polymer molecular MRI agents have been introduced with CEST MRI, wherein the CEST MRI images permit measurement of pH and the evaluation of its changes. In some embodiments, the polymer molecular MRI agents are introduced intravenously, intratumorally, and/or intraperitoneally into the subject.

In a seventh aspect, a method of using the polymer molecular MRI agents of the first aspect for theranostic imaging in a subject is described, said method comprising administering a bolus of a polymer molecular MRI agent of the first aspect to the subject with a known tumor, and imaging a region of the subject in proximity of the known tumor, wherein the polymer molecular MRI agent comprises a targeting molecule specific to a molecular target in the known tumor, and wherein the polymer molecular MRI agent further comprises a therapeutic agent. In some embodiments, the polymer molecular MRI agents are not substantially retained in non-cancerous organs or cells in vivo. In some embodiments, the bolus of the polymer molecular MRI agent is intravenously, intratumorally, or intraperitoneally injected into the subject. In some embodiments, the subject is imaged using MRI. In some embodiments, the subject is imaged using CEST MRI. In some embodiments, the region that the subject is imaged is in proximity of the known tumor site. In some embodiments, the therapeutic agent is a targeting molecule or is a signal-generating molecule. In some embodiments, the therapeutic agent is incorporated into the polymer molecular MRI agent along with the at least one targeting molecule or the at least one signal-generating molecule. In some embodiments, the therapeutic agent comprises a prodrug.

Advantageously, the polymer molecular MRI agents described herein comprise naturally occurring components and as such have minimal toxicity relative to MRI agents in the art. Further, by adjusting the at least one signal-generating molecule, the specificity and sensitivity of the polymer molecular MRI agents can be adapted, for example, to detect the molecular target of interest. The polymer molecular MRI agent described herein is readily translatable and can provide information on prostate-specific membrane antigen, a major target of cancer therapies, which can ultimately be used to accelerate drug development and to facilitate its evaluation in patients.

With regards to the obtainment of MRI sequences, in some embodiments, an 8-channel phase array surface coil can be used to maximize the signal-to-background at a tumor site. In some embodiments, slices for different sequences can be co-registered (slice thickness=1.5 mm) for within-subject comparison. In some embodiments, MR images can be generated using custom programs written in MatLab or ImageJ/FIJI. In some embodiments, Intensity can be quantified using Image J/FIJI.

With regards to the obtainment of CEST MRI images, in some embodiments, the parameters include TE=250 ms and TR=5 s, RARE factor=128, repetitions=43, B₁=1.2-4.8 μT, saturation time of 2-4 s with a frequency sweep from −12 to +12 ppm at 0.4 ppm increments. In some embodiments, to produce the CEST images, two sets of saturation images can be collected, a WASSR set for B₀ mapping and a CEST data set for characterizing contrast. These parameters were used for imaging with a 9.4T MRI scanner.

### EXAMPLE

Polymeric molecular MRI imaging agents were fabricated by conjugating the signal-generating molecules described in Table 1 and the targeting generating molecule (glutamate-ureido-lysine) to poly(acrylic acid) using EDC/NHS chemistry.

Specifically, 2100 MW PAA polymer, which contains ~29 acrylic acid sites, was dissolved in a 1:4 ratio water-DMSO solution (22.5 mg in 800 μL). EDC and NHS were added at a 1.5:1 and 3:1 mole ratio relative to the total number of acrylic acid sites in the PAA polymer and allowed to stir at room temperature (RT) for 5 minutes. The signal-generating molecule and/or target molecule was dissolved in a water-DMSO solution (<600 μL, ratio depending on solubility) at a 2:1 and 1:10 ratio relative to the number of acrylic acid sites. The solutions were combined, stirred at RT overnight, and then dialyzed using a 2,000 MW filter in ddH2O with multiple washes to remove impurities.

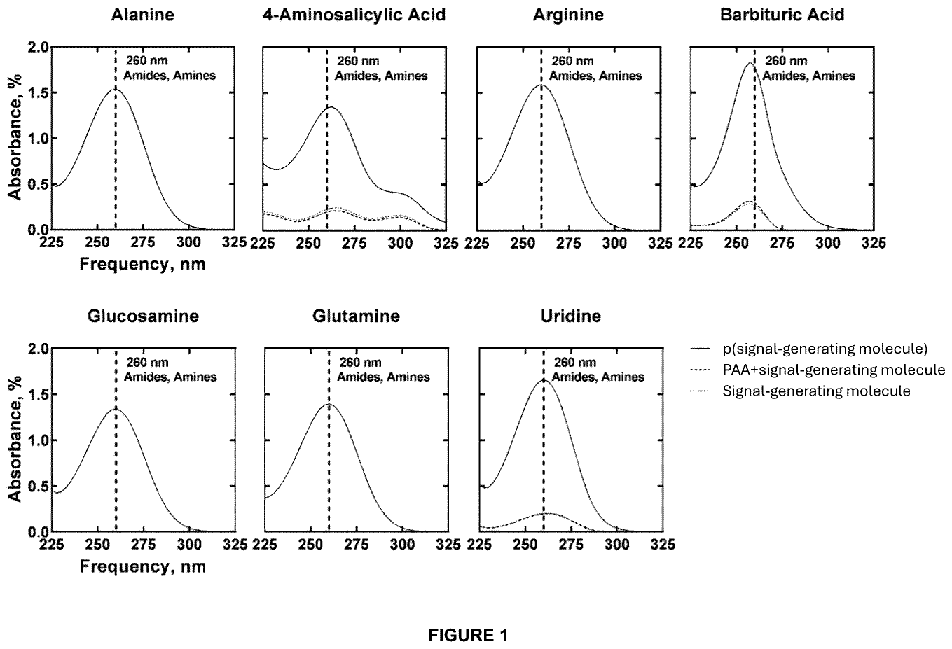

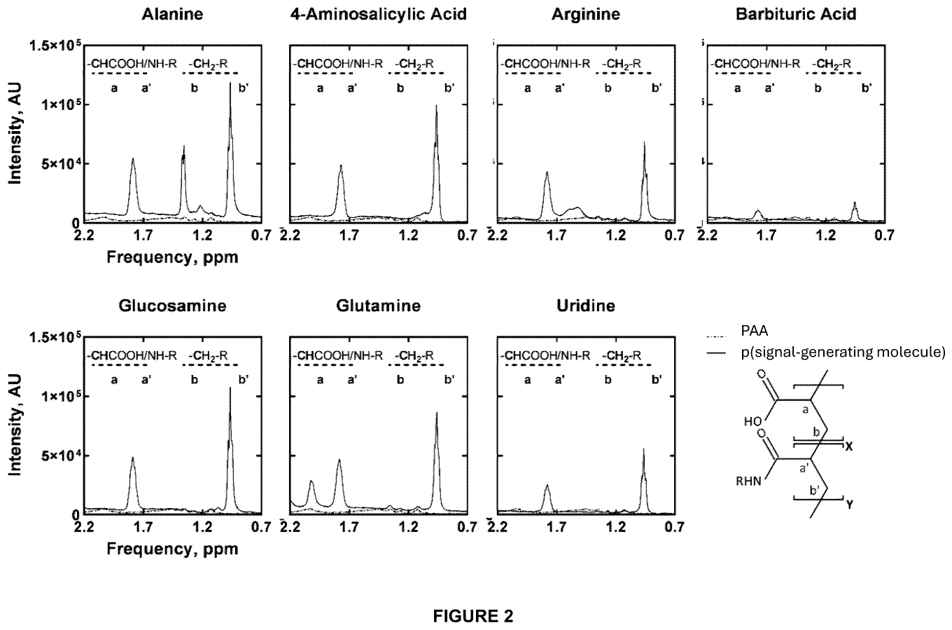

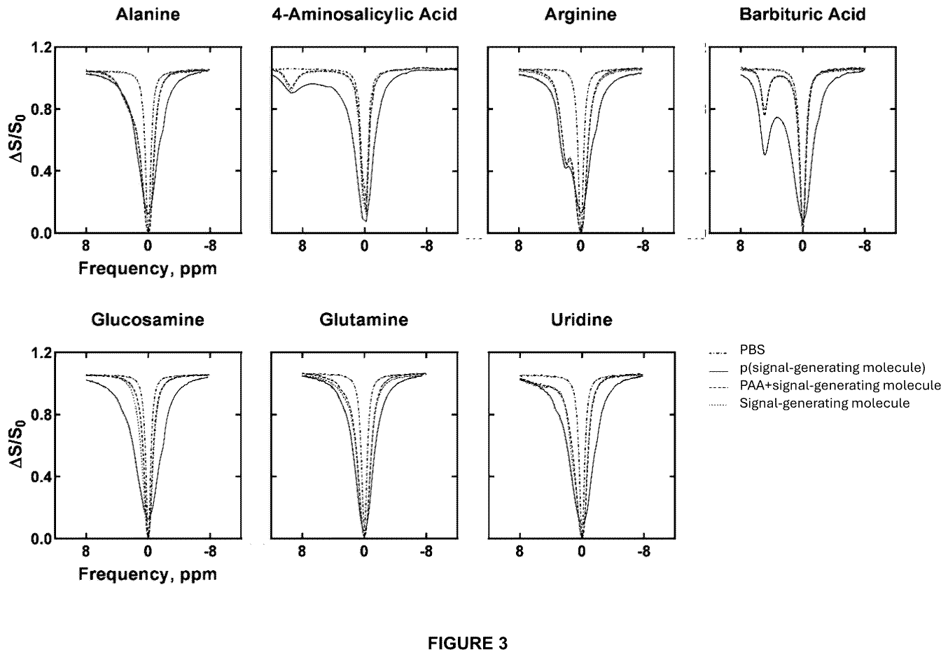

All the signal-generating molecules proposed could be conjugated to ~70% of the available sites in the polymer according to UV-VIS spectrometry signal (FIG. 1) and NMR chemical shifting (FIG. 2). The resulting CEST MRI agents (were visualized at a magnet field strength of 9.4 T, a field strength B₁ of 3.6 μT, and a saturation time tₛₐₜ of 3 s from a range of radiofrequencies (−2 ppm to +9 ppm) at pH 7.3 (FIG. 3). Abbreviations are defined in Table 1. Referring to FIG. 3, it can be seen that all polymer conjugates generated MRI contrast (S/S₀ in Z-spectra) compared to the PBS only control. Although not shown, the ΔS/S₀ in MTR_{asym} spectra also evidenced that all polymer conjugates generated MRI contrast relative to the PBS only control, wherein the so-called MTR, or magnetization transfer ratio, MTR=(S^{-Δω}−S^{+Δω})/S₀, is calculated with respect to the water signal at 0 ppm, and is the quantification of the magnetization transfer process. The MRI parameters for maximum contrast depended on the signal-generating molecule utilized, which permits simultaneous imaging of multiple targets.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

### REFERENCES

Banerjee S R, et al. Salicylic Acid-Based Polymeric Contrast Agents for Molecular Magnetic Resonance Imaging of Prostate Cancer. Chemistry—A European Journal. 2018; 24(28):7235-42. doi: doi.org/10.1002/chem.201800882.
Benešová M, et al. Linker Modification Strategies To Control the Prostate-Specific Membrane Antigen (PSMA)-Targeting and Pharmacokinetic Properties of DOTA-Conjugated PSMA Inhibitors. Journal of Medicinal Chemistry. 2016; 59(5):1761-75. doi: 10.1021/acs.jmedchem.5b01210.
Fragomeni R A S, et al. Imaging of Nonprostate Cancers Using PSMA-Targeted Radiotracers: Rationale, Current State of the Field, and a Call to Arms. Journal of Nuclear Medicine. 2018; 59(6):871-7. doi: 10.2967/jnumed. 117.203570.
García Garayoa E, et al. Influence of the Molecular Charge on the Biodistribution of Bombesin Analogues Labeled with the [99mTc(CO)3]-Core. Bioconjugate Chemistry. 2008; 19(12):2409-16. doi: 10.1021/bc800262m.
Han Z, et al. Extradomain-B Fibronectin-Targeted Dextran-Based Chemical Exchange Saturation Transfer Magnetic Resonance Imaging Probe for Detecting Pancreatic Cancer. Bioconjugate Chemistry. 2019; 30(5):1425-33. doi: 10.1021/acs.bioconjchem.9b00161.
Liu G, et al. A dextran-based probe for the targeted magnetic resonance imaging of tumours expressing prostate-specific membrane antigen. Nature Biomedical Engineering. 2017; 1(12):977-82. doi: 10.1038/s41551-017-0168-8.
Numasawa K, et al. A Fluorescent Probe for Rapid, High-Contrast Visualization of Folate-Receptor-Expressing Tumors In Vivo. Angewandte Chemie International Edition. 2020; 59(15):6015-20. doi: doi.org/10.1002/anie.201914826.
Szabo Z, et al. Initial Evaluation of [18F]DCFPyL for Prostate-Specific Membrane Antigen (PSMA)-Targeted PET Imaging of Prostate Cancer. Molecular Imaging and Biology. 2015; 17(4):565-74. doi: 10.1007/s11307-015-0850-8.
van Zijl P C, Yadav N N. Chemical exchange saturation transfer (CEST): what is in a name and what isn't? Magn Reson Med. 2011; 65(4):927-48. doi: 10.1002/mrm.22761. PubMed PMID: 21337419; PMCID: 3148076.
Wüstemann T, et al. Design of Internalizing PSMA-specific Glu-ureido-based Radiotherapeuticals. Theranostics. 2016; 6(8):1085-95. Epub 20160428. doi: 10.7150/thno.13448. PubMed PMID: 27279903; PMCID: PMC4893637.
Zanetti-Domingues L C, et al. Hydrophobic Fluorescent Probes Introduce Artifacts into Single Molecule Tracking Experiments Due to Non-Specific Binding. PLOS ONE. 2013; 8(9):e74200. doi: 10.1371/journal.pone.0074200.

1. A polymeric molecular MRI agent comprising a polymer, at least one signal-generating molecule, and optionally at least one targeting molecule.

2. The MRI agent of claim 1, wherein the at least one signal-generating molecule comprises alanine, 4-aminosalicyclic acid, arginine, barbituric acid, glucosamine, glutamine, uridine, or any combination thereof.

3. The MRI agent of claim 1, wherein the polymer is an acrylic-acid containing polymer.

4. The MRI agent of claim 1, wherein the polymer comprises poly(acrylic acid) (PAA).

5. The MRI agent of claim 4, wherein an efficiency of conjugation of at least one signal-generating molecule to a PAA polymer is at least about 60%.

6. The MRI agent of claim 1, wherein a mole ratio of the at least one signal-generating molecule to polymer is in a range from about 1:5 to about 1:60 when using PAA polymers having a MW in a range of about 1250 Da to about 20000 Da.

7. The MRI agent of claim 1, wherein the polymeric molecular MRI agents comprise only one type of signal-generating molecule conjugated to the polymer.

8. The MRI agent of claim 1, wherein the polymeric molecular MRI agents comprise a mixture of at least two or more of the signal-generating molecules conjugated to the polymer.

9. The MRI agent of claim 1, comprising at least one targeting molecule, wherein the at least one targeting molecule binds a molecular target of interest.

10. The MRI agent of claim 9, wherein the molecular target of interest is a cancer biomarker.

11. The MRI agent of claim 9, wherein the molecular target of interest is prostate-specific membrane antigen (PSMA) and the targeting molecule comprises glutamate-ureido-lysine (GUL).

12. The MRI agent of claim 9, wherein the molecular target of interest is a prostate-specific membrane antigen (PSMA), a programmed death-ligand 1 (PD-L1) biomarker, an arginine-glycine-aspartate (RGD)-binding integrin, or a Fibroblast Activation Protein-Alpha (FAP).

13. The MRI agent of claim 9, wherein the mole ratio of the at least one targeting molecule to polymer is in a range from about 1:1 to about 4:1.

14. The MRI agent of claim 1, further comprising at least one therapeutic agent, wherein the therapeutic agent is (i) a targeting molecule or is a signal-generating molecule or (ii) incorporated into the polymer molecular MRI agent along with the at least one targeting molecule and/or the at least one signal-generating molecule.

15. The MRI agent of claim 14, wherein the therapeutic agent comprises a prodrug.

16. A method of making the polymeric molecular MRI agent of claim 1, said method comprising:
dissolving a polymer in a first water-miscible solution to produce a first solution;
adding an amount of 1-ethyl-3-(3-dimethylaminopropyl)carbodiimide (EDC) and an amount of N-hydroxysuccinimide (NHS) to the first solution to produce a second solution;
dissolving at least one signal-generating molecule and at least one targeting molecule to a second water-miscible solution to produce a third solution; and
combining the second solution and the third solution,
wherein the at least one signal-generating molecule and the at least one targeting molecule are conjugated to the polymer using a carbodiimide reaction to produce the polymer molecular MRI agent.

17. A method of determining if a subject has a tumor, said method comprising administering a bolus of the polymer molecular MRI agent of claim 1 to a subject suspected of having a tumor, and imaging a region of the subject to determine if the polymer molecular MRI agent has been internalized and biodistributed in cells of the tumor, wherein the polymer molecular MRI agent comprises a targeting molecule specific to a molecular target in the cells of the tumor.

18. A method of monitoring a change in size of a known tumor in a subject, said method comprising administering a bolus of the polymer molecular MRI agent of claim 1 to the subject with the known tumor, and imaging a region of the subject in proximity of the known tumor to determine if the tumor has increased or decreased in size, wherein the polymer molecular MRI agent comprises a targeting molecule specific to a molecular target in the known tumor.

19. A method of using the polymer molecular MRI agents of claim 1 for measurement of pH and evaluation of pH changes, in vivo, wherein said method comprises introducing (i) multiple different polymer molecular MRI agents whose pH sensitivity for signal generation differ or (ii) polymer molecular MRI agents with multiple different signal-generating molecules with differing pH sensitivity to a subject, and imaging a region in proximity of where the polymer molecular MRI agents have been introduced with chemical exchange saturation transfer (CEST) MRI, wherein the CEST MRI images permit measurement of pH and the evaluation of its changes.

20. A method of using the polymer molecular MRI agents of claim 1 for theranostic imaging in a subject, said method comprising administering a bolus of the polymer molecular MRI agent to the subject with a known tumor, and imaging a region of the subject in proximity of the known tumor, wherein the polymer molecular MRI agent comprises a targeting molecule specific to a molecular target in the known tumor.
